(19) United States
(12) Patent Application Publication
(10) Pub. No.: US 2023/0033993 A1
(43) Pub. Date: Feb. 2, 2023

(54) PHOTOTHERAPY FACE MASK

(71) Applicant: THE LOTUS GROUP, INC., dba GLOBALMED TECHNOLOGIES, Napa, CA (US)

(72) Inventors: Sanders Ergas, Napa, CA (US); Quinten Stanier, Napa, CA (US); Addison Ergas, Napa, CA (US); Layne Ergas, San Diego, CA (US)

(21) Appl. No.: 17/874,054

(22) Filed: Jul. 26, 2022

Related U.S. Application Data
(60) Provisional application No. 63/226,153, filed on Jul. 27, 2021.

(51) Int. Cl. A61N 5/06

(52) U.S. Cl. CPC A61N 5/0616; A61N 2005/0647; A61N 2005/0651

(57) ABSTRACT

A phototherapy mask for phototherapy treatment of a user. The phototherapy mask includes a backing layer, an electronics layer, a skin-contacting layer and fastening means. The phototherapy mask is adapted to fold about the face of the user in a first plane and shaped to releasably fasten about the head of the user using the fastening means. The phototherapy mask includes at least one facial treatment region adapted to fold in a second plane relative to the first plane.

### RELATED APPLICATION

This application claims priority to U.S. Provisional patent application Ser. No. 63/226,153, filed Jul. 27, 2021, the entire disclosure of which is hereby incorporated by reference in its entirety.

### TECHNICAL FIELD

The present invention generally relates to phototherapy devices. More specifically, it relates to phototherapy devices for a user's face employing Light Emitting Diodes (LEDs).

### BACKGROUND OF THE INVENTION

Phototherapy is the use of light to treat medical conditions such as psoriasis, atopic eczema and acne. The broad term “phototherapy” includes ultraviolet phototherapy, chromatotherapy (or colored light therapy), photopheresis, extracorporeal photochemotherapy, and photodynamic therapy.

In the prior art, home use facial phototherapy devices typically comprise a facial mask having an array of LED emitters and configured as either as a rigidly formed contoured facial shape which is placed over the face of a user, or a flat flexible sheet which is wrapped about the head of a user.

Shaped phototherapy masks typically offer poor fitting due to the variance in human head shape and size, are often complex to manufacture and require large shipping cartons (due to their shape) and a high associated shipping cost. Flat masks allow for simple manufacture, relatively low shipping costs (due to their low profile) and general ease of use by an end user due to the ease of adjustment.

Flat masks are problematic due to the difficulty matching a two-dimensional flat sheet to a user's three-dimensional face. In particular, a flat sheet is incapable of contouring about the chin of a user and therefore treatment of this region is at best inadequate and most likely not possible.

It is an object of this invention to provide an improved phototherapy mask which solves this problem while maintaining the benefits of having a flat flexible shape.

### SUMMARY

In an embodiment, a phototherapy mask is provided for phototherapy treatment of a user. The phototherapy mask comprises a backing layer made of a flexible material; an electronics layer; a skin-contacting layer; and fastening means. The phototherapy mask is adapted to fold about the face of the user in a first plane and shaped to releasably fasten about the head of the user using the fastening means. The phototherapy mask includes at least one facial treatment region adapted to fold in a second plane relative to the first plane.

In another embodiment, a phototherapy mask is provided for phototherapy treatment of a user. The phototherapy mask comprises a backing layer; an electronics layer; a skin-contacting layer; and fastening means configured to releasably fasten the phototherapy mask about the head of the user.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

FIG. 1 is a front view of a phototherapy face mask according to an embodiment of the present invention, as worn by a user;

FIG. 2 is a side perspective view of the phototherapy face mask of FIG. 1, as worn by a user;

FIG. 3 is a cross-sectional view of the phototherapy face mask of FIG. 1;

FIG. 4 is a front view of a first layer of the phototherapy face mask of FIG. 1;

FIG. 5 is a front view of a second layer of the phototherapy face mask of FIG. 1;

FIG. 6 is a front view of a third layer of the phototherapy face mask of FIG. 1;

FIG. 7 is a rear view of the phototherapy face mask of FIG. 1 with related elements;

FIG. 8 is a front view of the phototherapy face mask of FIG. 1;

FIG. 9 is a right side view of the phototherapy face mask of FIG. 1;

FIG. 10 is a left side view of the phototherapy face mask of FIG. 1;

FIG. 11 is a top plan view of the phototherapy face mask of FIG. 1;

FIG. 12 is a bottom plan view of the phototherapy face mask of FIG. 1; and

FIG. 13 is a top rear perspective view of the phototherapy face mask of FIG. 1.

### DETAILED DISCLOSURE

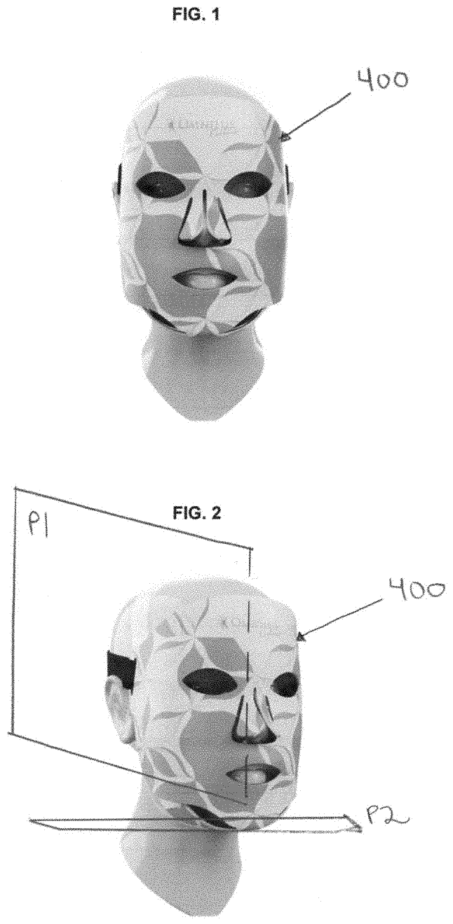

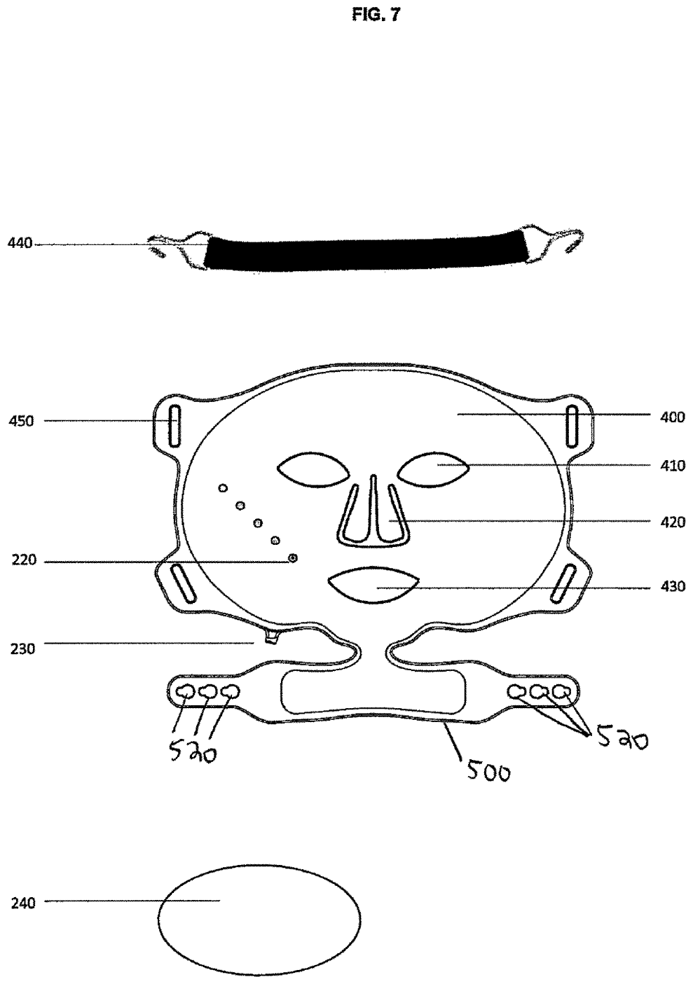

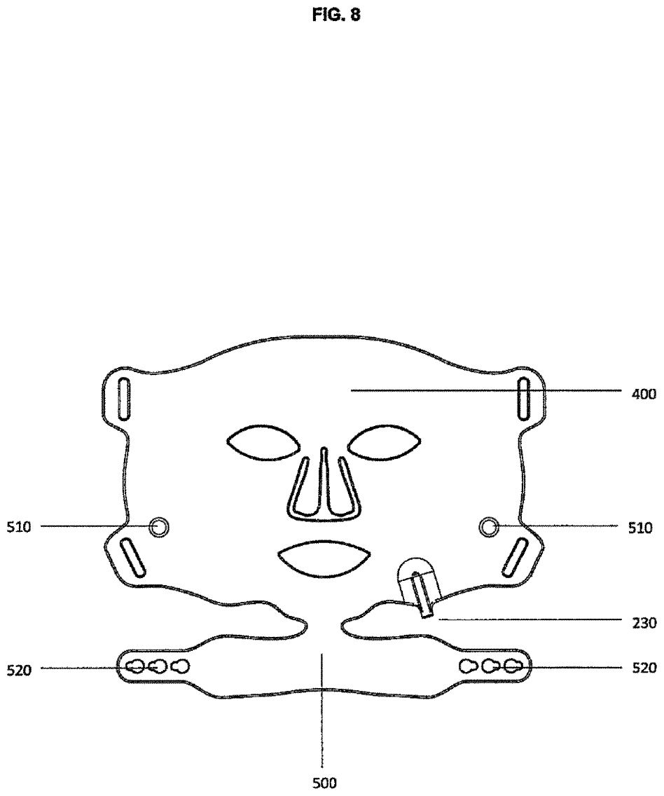

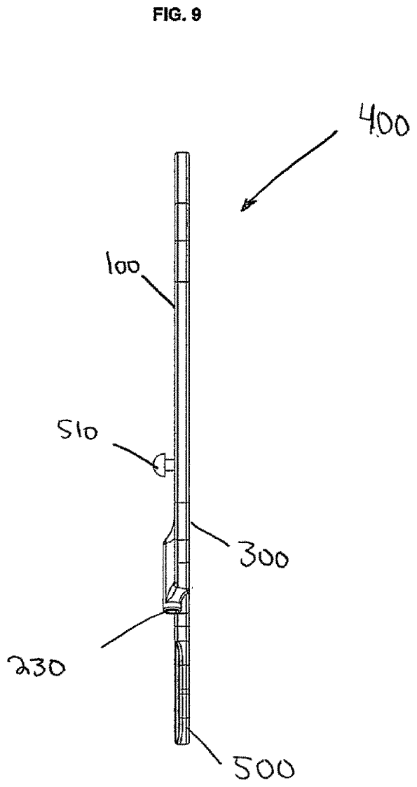

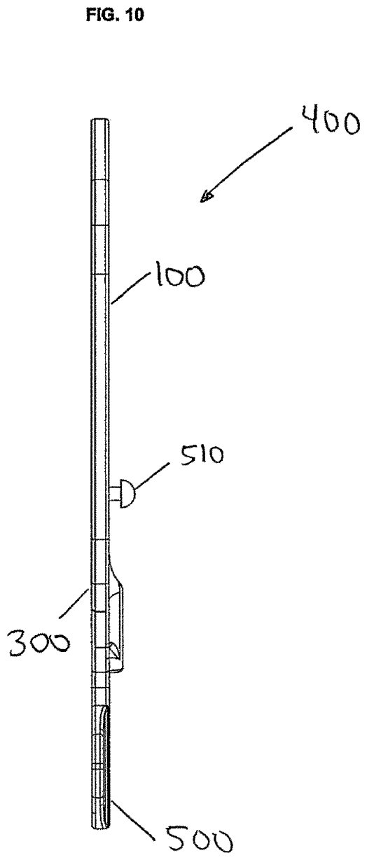

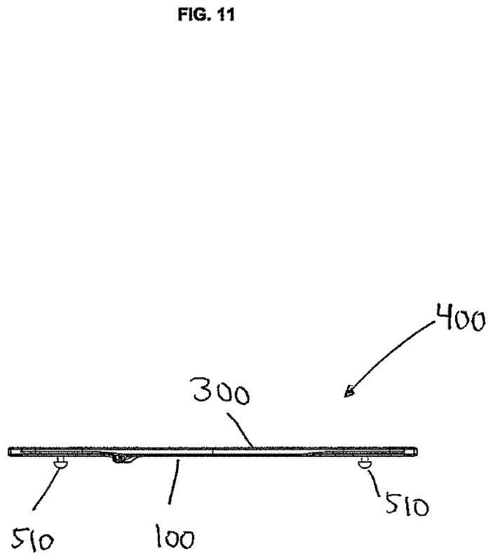

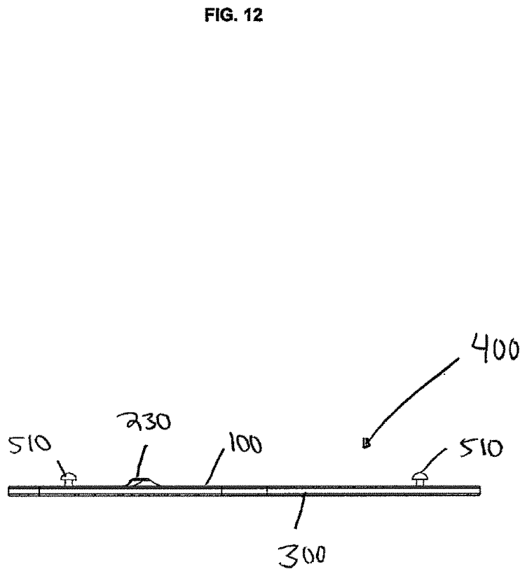

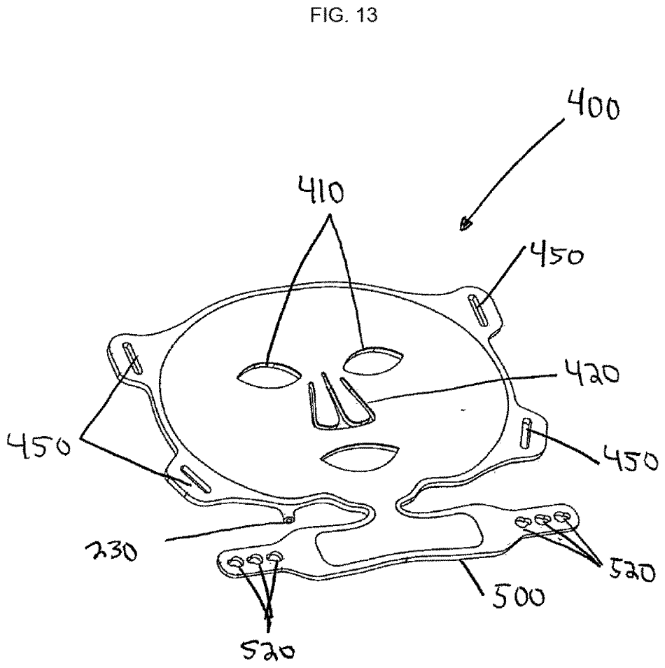

Embodiments of phototherapy face masks of the present invention are described herein and shown in FIGS. 1-13

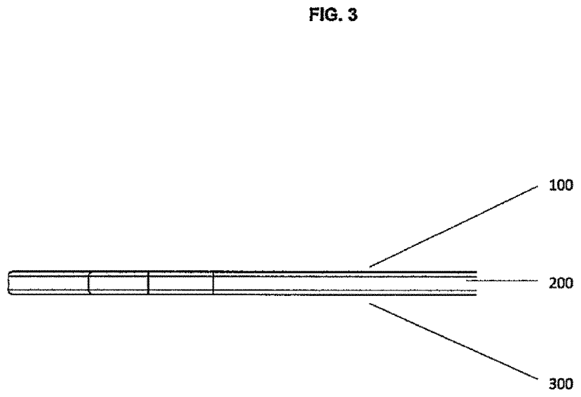

The phototherapy mask 400 is configured for use in the phototherapy treatment of a user's face and chin area. As shown in FIG. 3, the phototherapy mask 400 includes a first layer 100 that constitutes a backing layer, a second layer 200 that constitutes an electronics layer, and a third layer 300 that constitutes a skin-contacting layer. These layers are discussed below.

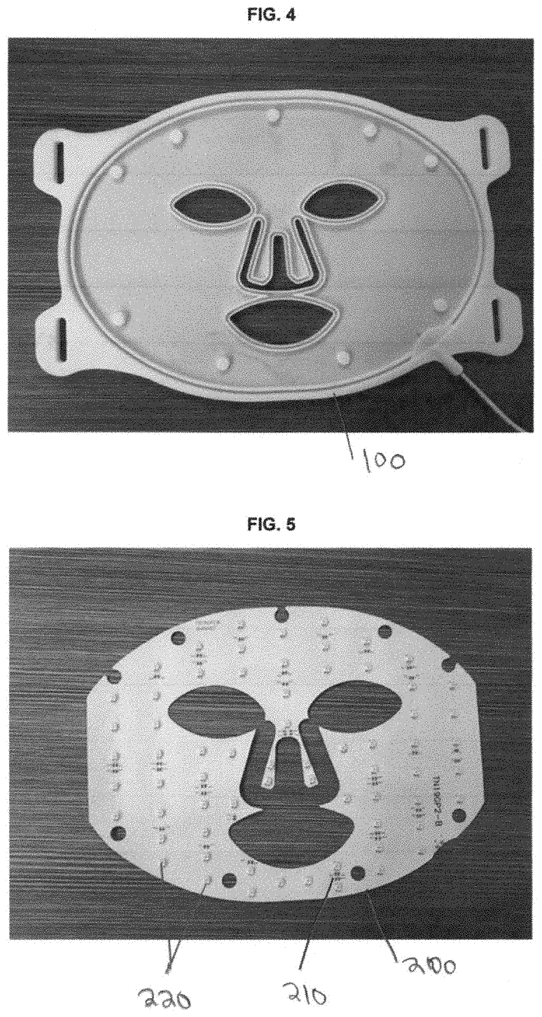

The backing (i.e., first) layer 100 is shown in FIG. 4. The backing layer is made from a flexible material. In various preferred embodiments, the flexible material is silicone.

The electronics (i.e., second) layer 200 is shown in FIG. 5. The electronics layer 200 includes a flexible printed circuit board (PCB) 210 and one or more light sources 220. The flexible printed circuit board 210 contains a flexible circuit that is laid out so that it is designed to flex in the intended direction. For example, the flexible circuit is typically designed to flex mainly in one direction only. The flexible circuit consists of a thin metallic layer of traces (usually copper) that is bonded to a dielectric layer (usually polyimide).

In preferred embodiments, tracks of the flexible PCB are located away from a known bend point of the mask 400. A track on a PCB is a conductive path or the running path of copper (or other metal) that runs all over the circuit board, acting as a connection between two points on the PCB. The conductive path connects two different components in the PCB. The bend point is the region on the mask 400 that significantly deforms under normal use when a mask is contoured around a face. Typically, such deformation is greater than an angle of 60° from the normal. For example the narrowed neck region between the round facial area and the under-chin area deforms by approximately 90° when contoured under the chin.

In various embodiments, the light sources 220 are LEDs capable of emitting light with at least one wavelength between 300 nm and 1200 nm. In various preferred embodiments, the LEDs are capable of emitting light with at least one wavelength between 420 nm and 1080 nm. In various embodiments, the light sources include an array of two or more LEDs emitting light in two or more wavelengths simultaneously. The light sources are configured to emit light in the direction of the skin-contacting layer 300.

Referring to FIG. 8, the electronic layer 200 further includes an electrical connector 230 configured to electrically connect to a remote power controller unit 240. The power controller unit 240 is adapted to provide power to the electronic layer 200 and to control the light sources 220 therein. In alternate embodiments, the power controller unit and/or a power source are not remote, but are integral to the electronic layer.

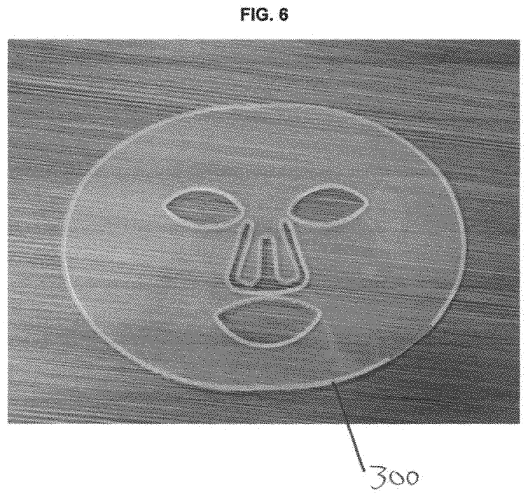

The skin-contacting (i.e., third) layer 300 is shown in FIG. 6. The skin-contacting layer 300 is made from a flexible material. In various preferred embodiments, the flexible material is silicone. The skin-contacting layer 300 is adapted to allow for the transmission of light from the electronic layer 200 to the skin of a user. In preferred embodiments, the skin-contacting layer 300 is formed from a material that is translucent or transparent. In an alternate embodiment of the invention, the skin-contacting layer 300 is opaque but is configured to have transparent regions coinciding with the location of the light sources on the electronic layer 200. In various embodiments, the transparent regions may be apertures, openings or holes, or the transparent regions may alternately include regions of different material with good light transmission properties, for example transparent windows. These regions may be obtained by overmoulding during production a second transparent material alongside the backing layer in one non-limiting example.

The phototherapy mask 400 includes apertures for the eyes 410, nose 420 and mouth 430 of a user to allow for comfortable use.

The phototherapy mask 400 is adapted to fold about the face of a user in a first plane P1 (see FIG. 2) and shaped to releasably fasten about the head of a user. In various embodiments, the mask 400 is fastened to the user's head using one or more fixing straps 440 and cooperating retainer loops 450 formed in the mask (see FIG. 8). The fixing straps 440 may be elasticated and/or may fasten by means of a fastening clip, hook and loop, buckle or other fastening means capable of releasably connecting to/cooperating with the retainer loops 450 to secure the mask 400 to the user's face.

The phototherapy mask 400 is further configured to provide at least one facial treatment region 500 that is adapted to fold in a second plane P2 relative to the first plane of the facial region (see FIG. 2). In the preferred embodiment the at least one facial treatment region 500 is configured to fold under the chin of a user.

The facial treatment region 500 is adapted to releasably affix to the backing layer 100 by one or more fastening means 510 formed therein. In various embodiments, these fastening means may include a fastening clip, buckle, button, peg, protrusion, anchor, clasp, hook, loop closure or any other mechanical means for affixing the facial treatment region 500 to the backing layer 100. In preferred embodiments the fastening means 510 is adjustable to allow for a range of user face sizes. Preferably, the fastening means 510 is configured to removably (e.g., insertably) connect to one or more apertures 520 in the facial treatment region 500 to affix the facial treatment region 500 to the fastening means 510 of the backing layer 100.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A phototherapy mask for phototherapy treatment of a user, comprising:
a backing layer made of a flexible material;
an electronics layer;
a skin-contacting layer; and
fastening means;
wherein the phototherapy mask is adapted to fold about the face of the user in a first plane and shaped to releasably fasten about the head of the user using the fastening means; and
wherein the phototherapy mask includes at least one facial treatment region adapted to fold in a second plane relative to the first plane.

2. The phototherapy mask of claim 1, wherein the flexible material comprises silicone.

3. The phototherapy mask of claim 1, wherein the electronics layer includes a flexible printed circuit board and one or more light sources.

4. The phototherapy mask of claim 3, wherein the one or more light sources are light-emitting diodes capable of emitting light with at least one wavelength between 300 nm and 1200 nm.

5. The phototherapy mask of claim 4, where the at least one wavelength is between 420 nm and 1080 nm.

6. The phototherapy mask of claim 3, wherein the electronic layer further includes an electrical connector configured to electrically connect to a power controller unit that is adapted to provide power to the electronic layer and to control the light sources therein.

7. The phototherapy mask of claim 6, wherein the power controller unit is remote from the phototherapy mask.

8. The phototherapy mask of claim 6, wherein the power controller unit is integral to the electronic layer.

9. The phototherapy mask of claim 1, wherein the at least one facial treatment region is configured to fold under a user's chin.

10. The phototherapy mask of claim 1, wherein the fastening means includes one or more fixing straps and cooperating retainer loops.

11. A phototherapy mask for phototherapy treatment of a user, comprising:
a backing layer;
an electronics layer;
a skin-contacting layer; and
fastening means configured to releasably fasten the phototherapy mask about the head of the user.

12. The phototherapy mask of claim 11, where the backing layer comprises a flexible material.

13. The phototherapy mask of claim 12, where the flexible material is silicone.

14. The phototherapy mask of claim 14, where the electronics layer includes a flexible printed circuit board and one or more light sources.

15. The phototherapy mask of claim 14, where the one or more light sources are light emitting diodes capable of emitting light with at least one wavelength between 300 nm and 1200 nm.

16. The phototherapy mask of claim 15, where the at least one wavelength is between 420 nm and 1080 nm.

17. The phototherapy mask of claim 14, wherein the electronic layer further includes an electrical connector configured to electrically connect to a power controller unit that is adapted to provide power to the electronic layer and to control the light sources therein.

18. The phototherapy mask of claim 11, wherein the phototherapy mask is adapted to fold about the face of the user in a first plane and shaped to releasably fasten about the head of the user using the fastening means; and
wherein the phototherapy mask includes at least one facial treatment region adapted to fold in a second plane relative to the first plane.

19. The phototherapy mask of claim 18, wherein the at least one facial treatment region is configured to fold under a user's chin.

20. The phototherapy mask of claim 11, wherein the fastening means includes one or more fixing straps and cooperating retainer loops.
